(19) United States
(12) Patent Application Publication
(10) Pub. No.: US 2023/0039028 A1
(43) Pub. Date: Feb. 9, 2023

(54) METHODS OF SUPPRESSING MICROGLIAL ACTIVATION

(71) Applicant: The Brigham and Women`s Hospital, Inc., Boston, MA (US)

(72) Inventors: Oleg Butovsky, Boston, MA (US); Howard L. Weiner, Brookline, MA (US)

(21) Appl. No.: 17/698,617

(22) Filed: Mar. 18, 2022

Related U.S. Application Data
(63) Continuation of application No. 16/619,866, filed on Dec. 5, 2019, now abandoned.
(60) Provisional application No. 62/515,711, filed on Jun. 6, 2017.

(51) Int. Cl. C07K 16/28

(52) U.S. Cl. CPC C07K 16/2809; A61K 2039/505

(57) ABSTRACT

The present invention provides methods of suppressing the activation of microglial cells, methods of ameliorating or treating the neurological effects of cerebral ischemia or cerebral inflammation, methods of ameliorating or treating specific diseases that affect the CNS by administering an anti-CD3 antibody.

### CLAIM OF PRIORITY

This application is a continuation application of U.S. patent application Ser. No. 16/619,866, filed Dec. 5, 2019, which is a § 371 National Stage Application of PCT/US2018/036261, filed Jun. 6, 2018, and claims the benefit of U.S. Provisional Patent Application Serial No. 62/515,711, filed on Jun. 6, 2017. The entire contents of the foregoing are hereby incorporated by reference.

### SEQUENCE LISTING

The instant application contains a Sequence Listing which has been submitted electronically in ASCII format and is hereby incorporated by reference in its entirety. Said ASCII copy, created on Mar. 18, 2022, is named Sequence Listing.txt and is 9,790 bytes in size.

### FIELD OF THE INVENTION

The present invention relates to generally to methods of suppressing the activation of microglial cells, methods of ameliorating or treating the neurological effects of cerebral ischemia or cerebral inflammation, and methods of ameliorating or treating specific diseases that affect the CNS by administering an anti-CD3 antibody.

### BACKGROUND OF THE INVENTION

Human CD3 antigen consists of a minimum of four invariant polypeptide chains, which are non-covalently associated with the T-cell receptors on the surface of T-cells, and is generally now referred to as the CD3 antigen complex. It is intimately involved in the process of T-cell activation in response to antigen recognition by the T-cell receptors. Due to the fundamental nature of CD3 in initiating an anti-antigen response, monoclonal antibodies against this receptor have been proposed as being capable of blocking or at least modulating the immune process and thus as agents for the treatment of inflammatory and/or autoimmune disease.

The Central Nervous System (CNS) has long been considered to be a site of relative immune privilege. However, it is increasingly recognized that CNS tissue injury in acute and chronic neurological disease may be mediated by the CNS inflammatory response. The CNS inflammatory response is primarily mediated by inflammatory cytokines.

There is a need in the art for a more specific therapeutic targeting system to control microglial cell activation. In addition, there is a need for a method of inhibiting amyloid plaque formation in patients suffering from neurodegenerative disorders.

### SUMMARY OF THE INVENTION

In various aspects the disclosure provides method for decreasing microglial activation by contacting a microglial cell with an anti-CD3 antibody. The cell is contacted with the antibody in an amount sufficient to suppress the microglial inflammatory phenotype. For example, the cell is contacted with the antibody in an amount sufficient to reduce microglial expression of CD74 and/or H2-AB1 or increase the microglial expression of CX3CR1 and/or TGFβ-1. Alternatively, the cell is contacted with the antibody in an amount sufficient to increase Ly6C^{high} splenocyte expression of one or more of CX3CR1 CCR2, Hsp40 or Dusp1.

Also provided by the disclosure are methods of treating, preventing or alleviating a sign or symptom of a disease associated with microglial activation in a subject, by administering to a subject in need thereof an anti-CD3 antibody. The administration is oral or mucosal. Preferably the administration is intra-nasal.

A disease associated with microglial activation included for example, is a neurodegenerative disorder, an ischemic related disease or injury, traumatic brain injury or a lysosomal storage disease. A neurodegenerative disease is for example, Alzheimer's Disease, Parkinson's Disease, Amyotrophic Lateral Sclerosis (ALS), or Huntington's Disease. The ischemic related disease is a ischemic-reperfusion injury, stroke, myocardial infarction. For example, the ischemic-reperfusion injury is in lung tissue, cardiac, tissue and neuronal tissue. The traumatic brain injury is a concussion such as repetitive concussive injury or whiplash. The lysosomal storage disease is Neimann-Pick disease.

A sign or symptom of a disease associated with microglial activation is for example, amyloid plaque formation.

The anti-CD3 antibody is a monoclonal or polyclonal antibody.

For example, the anti-CD3 antibody is a fully human, humanized or chimeric.

An exemplary, e anti-CD3 antibody has a heavy chain complementarity determining region 1 (CDRH1) having the amino acid sequence GYGMH (SEQ ID NO: 1), a heavy chain complementarity determining region 2 (CDRH2) having the amino acid sequence VIWYDGSKKYYVDSVKG (SEQ ID NO: 3), a heavy chain complementarity determining region 3 (CDRH3) having the amino acid sequence QMGYWHFDL (SEQ ID NO: 4), a light chain complementarity determining region 1 (CDRL1) having the amino acid sequence RASQSVSSYLA (SEQ ID NO: 5), a light chain complementarity determining region 2 (CDRL2) having the amino acid sequence DASNRAT (SEQ ID NO: 6), and a light chain complementarity determining region 3 (CDRL3) having the amino acid sequence QQRSNWPPLT (SEQ ID NO: 7).

The anti-CD3 antibody has a variable heavy chain amino acid sequence having the amino acid sequence of SEQ ID NO: 8 and a variable light chain amino acid sequence having the amino acid sequence of SEQ ID NO: 9. Alternatively, the anti-CD3 antibody comprises a heavy chain amino acid sequence having the amino acid sequence of SEQ ID NO: 10 and a light chain amino acid sequence having the amino acid sequence of SEQ ID NO: 11.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIGS. 1A-B are a heatmap and a diagram, respectively, that show the effect of nasal anti-CD3 on microglia in old (24 month) wild type mice. FIG. 1A is a heatmap that depicts hierarchical clustering of 116 differentially expressed genes measured by RNA-sequencing analysis in FCRLS+ sorted microglia of 24 month old wild-type mice treated with anti-CD3 (clone 2C11) or isotype control. FIG. 1B is a diagram of ingenuity pathway analysis of differentially expressed genes which demonstrates that anti-CD3 suppresses the expression of IRF-7 driven inflammatory node in microglia from old mice.

FIG. 2 is a heatmap showing the effect of nasal anti-CD3 on microglia in young (2 month) wild type mice. The heatmap depicts hierarchical clustering of 210 differentially expressed genes measured by RNA-sequencing analysis in FCRLS+ sorted microglia of 2 month old wild type mice treated with anti-CD3 (clone 2C11) or isotype control.

FIGS. 3A-B are a heatmap and a series of nine graphs, respectively, showing that nasal anti-CD3 modulates the inflammatory phenotype of microglia cells in a mouse model of repetitive mild traumatic brain injury (TBI). Repetitive mild TBI (concussive injury) mice were treated with a closed head weight drop with rotational acceleration, using a 54 g weight and a 42″ drop, 1 injury daily for 5 straight days. Intranasal anti-CD3 or isotype control antibody (1 μg daily) was administered 1 hour following each injury, then daily for 6 additional days. TBI model mice were sacrificed 7 days following the last injury, their brains were harvested for myeloid cell isolation (Percoll), followed by fluorescence activated cell sorting (FACS). RNA was isolated from sorted microglia, then analyzed using Nanostring with the myeloid code set. FIG. 3A is a heatmap that depicts hierarchical clustering of differentially expressed genes in microglia isolated from TBI model mice treated with anti-CD3 or isotype control. FIG. 3B shows expression differences of 9 individual genes between anti-CD3 (black bars) treated TBI mice and isotype control (white bars) treated TBI mice. Copy number is depicted on the Y axis. Top row, from left to right: Adgrel, CX3CR1, INOS; middle row, from left to right: CD68, CCR2, Tgfbl; bottom row, from left to right: CD74, H2-Ab1 and TNF. * Heat map signifies the genes for which the number of copies was significantly different (P <0.05) between the anti-CD3 and isotype groups. CD74, the invariant chain involved in MHCII presentation, is downregulated in the anti-CD3 group. Similarly, H2-Ab1, one of the MHCII antigens, is also downregulated. CX3CR1 and TGFb1 (which has a role in inducing regulatory T cells) are both significantly increased in the anti-CD3 group.

FIGS. 4A-B are a heatmap and a series of 12 graphs, respectively, showing that nasal anti-CD3 modulated the inflammatory phenotype of splenic Ly6Chi monocytes. TBI mice were generated, treated and analyzed as described for FIG. 3. FIG. 4A is a heatmap that depicts hierarchical clustering of differentially expressed genes in splenic Ly6Chi monocytes isolated from TBI model mice treated with anti-CD3 or isotype control. FIG. 4B shows expression differences of 12 individual genes between anti-CD3 (black bars) treated TBI mice and isotype control (white bars) treated TBI mice. Copy number is depicted on the Y axis. Top row, from left to right: Adgrel, CX3CR1, INOS, Hsp40; middle row, from left to right: CD68, CCR2, Tgfbl, Duspl; bottom row, from left to right: CD74, H2-Ab1, TNF and Nod1. *Heat map signifies the genes for which the number of copies was significantly different (P<0.05) between the anti-CD3 and isotype groups. Both CX3CR1 and CCR2 are increased in the anti-CD3 group. Hsp40 (aka Dnajb6) and Duspl are both strongly increased in the anti-CD3 group. Hsp40 has been shown to be neuroprotective in CNS trauma, and Duspl is an anti-inflammatory molecule.

FIG. 5 is a diagram depicting the experimental design for anti-CD3 treatment of the APPPS1 amyloid-beta transgenic mouse model of Alzheimer's disease (AD). APPPS1 model mice were treated for 3 months with 1 μg/mouse of anti-CD3 (clone 2C11) intranasally every other day. At sacrifice, Clec7a+ microglia were sorted for transcriptomic analysis and brains were analyzed by confocal immunofluorescence.

FIG. 6 is a heatmap that demonstrates the hierarchical clustering of differentially expressed genes analyzed by RNA sequencing of Clec7a+ microglia from APPPS1 or wild type mice treated nasally with anti-CD3 or isotype control according to the experimental design depicted in FIG. 5. The clustering demonstrates that nasal anti-CD3 treatment modulated the transcriptomic profile of Clec7+ inflammatory microglia in APPPS1 transgenic mice as all anti-CD3 treated APPPS1 mice clustered together versus isotype control treatment. WT mice did not demonstrate clustering of anti-CD3 versus isotype control. However, WT versus APPPS1 AD mice clustered independently as would be expected. Nasal anti-CD3 modulates gene expression of Clec7+ microglia in APPPS1 mice but not littermate WT mice.

FIG. 7 is a series of 6 immunofluorescence confocal images of brains from WT (left), male APPPS1 (middle) and female APPPS1 mice (right) treated with nasal anti-CD3 (bottom row) or isotype control (top row). Human amyloid beta was stained blue, the homeostatic microglia marker P2Ry12 in green and the activation marker Clec7a in red. Male APPPS1 mice treated with anti-CD3 demonstrate less Clec7a plaque-associated microglia.

FIG. 8 is a diagram depicting the experimental design for anti-CD3 treatment of the P301S (Tau) transgenic mouse model for Alzheimer's disease. P301S (Tau transgenic mouse model) mice were treated for 2 months with 1 μg/mouse of anti-CD3 (clone 2C11) intranasally every other day. At sacrifice, Clec7a+ microglia were sorted for transcriptomic analysis.

FIG. 9 is a heatmap that demonstrates the hierarchical clustering of differentially expressed genes analyzed by RNA sequencing in P301S (Tau) transgenic mice treated nasally with anti-CD3 or isotype control according to the experimental design depicted in FIG. 8. The clustering demonstrates that nasal anti-CD3 treatment modulated the transcriptomic profile of Clec7+ inflammatory microglia in Tau transgenic mice as all anti-CD3 treated P301S mice clustered together versus isotype control treatment. These results demonstrate that nasal anti-CD3 can modulate the gene expression of Clec7+ microglia in Tau transgenic mice.

FIG. 10 is a series of 3 graphs depicting nasal anti-CD3 in cardiac Ischemia/Reperfusion. A mouse model of myocardial ischemia/reperfusion was treated with nasal anti-CD3. Mice were treated daily with a dose of 5 μg/mouse of anti-CD3 (aCD3, red squares) or isotype control (IC, black circles) starting from the time of injury to the end of the experiment. Anti-CD3 mice showed a beneficial effect as measured by percentage of fractional shortening (left graph), ejection fraction (middle graph) and fractional area change (right graph) compared to control naïve mice.

### DETAILED DESCRIPTION OF THE INVENTION

The methods and compositions described herein are based, in part, upon the discovery that the inflammatory phenotype of microglial cells is modulated by anti-CD3 antibodies. Specifically, it was discovered that CD74, the invariant chain involved in MHC II presentation and H2-AB1, a MHC II antigen is downregulated in microglia upon anti-CD3 administration. Critically, anti-CD3 administration not only modulates the gene expression of Clec7+ microglia in APPPS1 mice but also reduced the number of Clec730 plaque-associated microglia.

More specifically, the methods described herein relates to the inhibition of microglial activation by reducing CD3 expression.

Microglia are non-neuronal macrophage-like cells present in the developing and adult central nervous systems. Upon neuronal injury, microglia are transformed from a resting state to an activated state, characterized by changes in morphology, immunophenotype, migration, and proliferation. Activated microglia participate in the phagocytosis of neurons, and, furthermore, microglial proteases are involved in neuronal degradation

The present methods are useful in preventing, treating, or ameliorating neurological signs and symptoms associated with acute CNS injury. Acute CNS injury includes ischemic related disease. Ischemic related disease include for example, ischemic reperfusion injury (of lung, cardiac or neuronal tissue), stroke (caused by thrombosis, embolism or vasoconstriction), global cerebral ischemia (e.g., ischemia due to systemic hypotension of any cause, including cardiac infarction, cardiac arrhythmia, hemorrhagic shock, and post coronary artery bypass graft brain injury) and intracranial hemorrhage. Acute CNS injury also includes traumatic brain injury such as concussion (e.g., repetitive concussive injury), whiplash and closed head injury.

Further, the present methods and compounds are useful in preventing, treating, or ameliorating neurological signs and symptoms associated with chronic neurological disease, including but not limited to Alzheimer's disease (AD), Parkinson's Disease (PD), Huntington's Disease (HD), Amyotrophic Lateral Sclerosis (ALS), epilepsy, HIV-associated encephalopathy and AIDS related dementia.

The present methods and compounds are also useful in preventing, treating, or ameliorating the neurological signs and symptoms associated with lysomal storage diseases affecting the nervous system including the CNS, including but not limited to Neiman-Pick disease.

The present methods are also useful in preventing, treating, or ameliorating the neurological signs and symptoms associated with inflammatory conditions affecting the nervous system including the CNS, including but not acute disseminated encephalomyelitis.

Stated in a different way, the present methods and compounds are useful in preventing, suppressing or reducing the activation of microglia in the CNS that occurs as a part of acute or chronic CNS disease. The suppression or reduction of microglial activation can be assessed by various methods as would be apparent to those in the art; one such method is to measure the production or presence of compounds that are known to be produced by activated microglia, and compare such measurements to levels of the same compounds in control situations. Alternatively, the effects of the present methods and compounds in suppressing, reducing or preventing microglial activation may be assessed by comparing the signs and/or symptoms of CNS disease in treated and control subjects, where such signs and/or symptoms are associated with or secondary to activation of microglia.

Pathological hallmarks of Alzheimer's disease (AD) include extracellularly accumulated amyloid β (Aμ) plaques and intracellular neurofibrillary tangles in the brain. Activated microglia, are also found surrounding Aβ plaques. The study of the brain of AD mouse models revealed that Aβ plaque formation is completed by the consolidation of newly generated plaque clusters in vicinity of existed plaques. Activated microglia surrounding Aβ plaques take up Aβ, which are clusters developed inside activated microglia in vivo and this is followed by microglial cell death. These dying microglia release the accumulated Aβ into the extracellular space, which contributes to Aβ plaque growth. Thus, activated microglia can contribute to formation and growth of Aβ plaques by causing microglial cell death in the brain. Thus, the present methods and compounds are also useful in preventing, treating, or ameliorating diseases and disorders associated with the accumulation of amyloid β (Aβ) plaques.

As used herein, the terms “combating”, “treating” and “ameliorating” are not necessarily meant to indicate a reversal or cessation of the disease process underlying the CNS condition afflicting the subject being treated. Such terms indicate that the deleterious signs and/or symptoms associated with the condition being treated are lessened or reduced, or the rate of progression is reduced, compared to that which would occur in the absence of treatment. A change in a disease sign or symptom may be assessed at the level of the subject (e.g., the function or condition of the subject is assessed), or at a tissue or cellular level (e.g., the production of markers of glial activation is lessened or reduced). Where the methods of the present invention are used to treat chronic CNS conditions (such as Alzheimer's disease), the methods may slow or delay the onset of symptoms such as dementia, while not necessarily affecting or reversing the underlying disease process.

Anti-CD3 Antibodies

The anti-CD3 antibodies can be any antibodies specific for CD3. The term “antibody” as used herein refers to an immunoglobulin molecule or immunologically active portion thereof, i.e., an antigen-binding portion. Examples of immunologically active portions of immunoglobulin molecules include F(ab) and F(ab′).sub.2 fragments, which retain the ability to bind CD3. Such fragments can be obtained commercially, or using methods known in the art. For example, F(ab)₂ fragments can be generated by treating the antibody with an enzyme such as pepsin, a non-specific endopeptidase that normally produces one F(ab)₂ fragment and numerous small peptides of the Fc portion. The resulting F(ab)² fragment is composed of two disulfide-connected Fab units. The Fc fragment is extensively degraded and can be separated from the F(ab)₂ by dialysis, gel filtration or ion exchange chromatography. F(ab) fragments can be generated using papain, a non-specific thiol-endopeptidase that digests IgG molecules, in the presence of a reducing agent, into three fragments of similar size: two Fab fragments and one Fc fragment. When Fc fragments are of interest, papain is the enzyme of choice because it yields a 50,00 Dalton Fc fragment; to isolate the F(ab) fragments, the Fc fragments can be removed, e.g., by affinity purification using protein A/G. A number of kits are available commercially for generating F(ab) fragments, including the ImmunoPure IgG1 Fab and F(ab′)₂. Preparation Kit (Pierce Biotechnology, Rockford, Ill.). In addition, commercially available services for generating antigen-binding fragments can be used, e.g., Bio Express, West Lebanon, N.H.

The antibody can be a polyclonal, monoclonal, recombinant, e.g., a chimeric, de-immunized or humanized, fully human, non-human, e.g., murine, single chain antibody or single domain antibody. In some embodiments the antibody has effector function and can fix complement. In some embodiments, the antibody has reduced or no ability to bind an Fc receptor. For example, the anti-CD3 antibody can be an isotype or subtype, fragment or other mutant, which does not support binding to an Fc receptor, e.g., it has a mutagenized or deleted Fc receptor binding region. The antibody can be coupled to a toxin or imaging agent.

A number of anti-CD3 antibodies are known, including but not limited to OKT3 (muromonab/Orthoclone OKT3.TM., Ortho Biotech, Raritan, N.J.; U.S. Pat. No. 4,361,549); hOKT3(1 (Herold et al., N.E.J.M. 346(22):1692-1698 (2002); HuM291 (Nuvion™, Protein Design Labs, Fremont, Calif.); gOKT3-5 (Alegre et al., J. Immunol. 148(11):3461-8 (1992); 1F4 (Tanaka et al., J. Immunol. 142:2791-2795 (1989)); G4.18 (Nicolls et al., Transplantation 55:459-468 (1993)); 145-2C11 (Davignon et al., J. Immunol. 141(6):1848-54 (1988)); and as described in Frenken et al., Transplantation 51(4):881-7 (1991); U.S. Pat. Nos. 6,491,9116, 6,406,696, and 6,143,297).

Methods for making such antibodies are also known. A full-length CD3 protein or antigenic peptide fragment of CD3 can be used as an immunogen, or can be used to identify anti-CD3 antibodies made with other immunogens, e.g., cells, membrane preparations, and the like, e.g., E rosette positive purified normal human peripheral T cells, as described in U.S. Pat. Nos. 4,361,549 and 4,654,210. The anti-CD3 antibody can bind an epitope on any domain or region on CD3.

Chimeric, humanized, de-immunized, or completely human antibodies are desirable for applications which include repeated administration, e.g., therapeutic treatment of human subjects.

Chimeric antibodies contain portions of two different antibodies, typically of two different species. Generally, such antibodies contain human constant regions and variable regions from another species, e.g., murine variable regions. For example, mouse/human chimeric antibodies have been reported which exhibit binding characteristics of the parental mouse antibody, and effector functions associated with the human constant region. See, e.g., Cabilly et al., U.S. Pat. No. 4,816,567; Shoemaker et al., U.S. Pat. No. 4,978,745; Beavers et al., U.S. Pat. No. 4,975,369; and Boss et al., U.S. Pat. No. 4,816,397, all of which are incorporated by reference herein. Generally, these chimeric antibodies are constructed by preparing a genomic gene library from DNA extracted from pre-existing murine hybridomas (Nishimura et al., Cancer Research, 47:999 (1987)). The library is then screened for variable region genes from both heavy and light chains exhibiting the correct antibody fragment rearrangement patterns. Alternatively, cDNA libraries are prepared from RNA extracted from the hybridomas and screened, or the variable regions are obtained by polymerase chain reaction. The cloned variable region genes are then ligated into an expression vector containing cloned cassettes of the appropriate heavy or light chain human constant region gene. The chimeric genes can then be expressed in a cell line of choice, e.g., a murine myeloma line. Such chimeric antibodies have been used in human therapy.

Humanized antibodies are known in the art. Typically, “humanization” results in an antibody that is less immunogenic, with complete retention of the antigen-binding properties of the original molecule. In order to retain all the antigen-binding properties of the original antibody, the structure of its combining-site has to be faithfully reproduced in the “humanized” version. This can potentially be achieved by transplanting the combining site of the nonhuman antibody onto a human framework, either (a) by grafting the entire nonhuman variable domains onto human constant regions to generate a chimeric antibody (Morrison et al., Proc. Natl. Acad. Sci., USA 81:6801 (1984); Morrison and Oi, Adv. Immunol. 44:65 (1988) (which preserves the ligand-binding properties, but which also retains the immunogenicity of the nonhuman variable domains); (b) by grafting only the nonhuman CDRs onto human framework and constant regions with or without retention of critical framework residues (Jones et al. Nature, 321:522 (1986); Verhoeyen et al., Science 239:1539 (1988)); or (c) by transplanting the entire nonhuman variable domains (to preserve ligand-binding properties) but also “cloaking” them with a human-like surface through judicious replacement of exposed residues (to reduce antigenicity) (Padlan, Molec. Immunol. 28:489 (1991)).

Humanization by CDR grafting typically involves transplanting only the CDRs onto human fragment onto human framework and constant regions. Theoretically, this should substantially eliminate immunogenicity (except if allotypic or idiotypic differences exist). However, it has been reported that some framework residues of the original antibody also need to be preserved (Riechmann et al., Nature 332:323 (1988); Queen et al., Proc. Natl. Acad. Sci. USA 86:10,029 (1989)). The framework residues which need to be preserved can be identified by computer modeling. Alternatively, critical framework residues may potentially be identified by comparing known antibody combining site structures (Padlan, Molec. Immun. 31(3):169-217 (1994)). The invention also includes partially humanized antibodies, in which the 6 CDRs of the heavy and light chains and a limited number of structural amino acids of the murine monoclonal antibody are grafted by recombinant technology to the CDR-depleted human IgG scaffold (Jones et al., Nature 321:522-525 (1986)).

Deimmunized antibodies are made by replacing immunogenic epitopes in the murine variable domains with benign amino acid sequences, resulting in a deimmunized variable domain. The deimmunized variable domains are linked genetically to human IgG constant domains to yield a deimmunized antibody (Biovation, Aberdeen, Scotland).

The anti-CD3 antibody can also be a single chain antibody. A single-chain antibody (scFV) can be engineered (see, for example, Colcher et al., Ann. N.Y. Acad. Sci. 880:263-80 (1999); and Reiter, Clin. Cancer Res. 2:245-52 (1996)). The single chain antibody can be dimerized or multimerized to generate multivalent antibodies having specificities for different epitopes of the same target CD3 protein. In some embodiments, the antibody is monovalent, e.g., as described in Abbs et al., Ther. Immunol. 1(6):325-31 (1994), incorporated herein by reference.

Exemplary anti-CD3 antibodies, comprise a heavy chain complementarity determining region 1 (CDRH1) comprising the amino acid sequence GYGMH (SEQ ID NO: 1), a heavy chain complementarity determining region 2 (CDRH2) comprising the amino acid sequence VIWYDGSKKYYVDSVKG (SEQ ID NO: 3), a heavy chain complementarity determining region 3 (CDRH3) comprising the amino acid sequence QMGYWHFDL (SEQ ID NO: 4), a light chain complementarity determining region 1 (CDRL1) comprising the amino acid sequence RASQSVSSYLA (SEQ ID NO: 5), a light chain complementarity determining region 2 (CDRL2) comprising the amino acid sequence DASNRAT (SEQ ID NO: 6), and a light chain complementarity determining region 3 (CDRL3) comprising the amino acid sequence QQRSNWPPLT (SEQ ID NO: 7).

In some embodiments, the anti-CD3 antibody comprises a variable heavy chain amino acid sequence comprising

(SEQ ID NO: 8)
QVQLVESGGGVVQPGRSLRLSCAASGFKFSGYGMHWVRQAPGKGLEWVAV
IWYDGSKKYYVDSVKGRFTISRDNSKNTLYLQMNSLRAEDTAVYYCARQM
GYWHFDLWGRGTLVTVSS

and a variable light chain amino acid sequence comprising

(SEQ ID NO: 9)
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPPLTFG
GGTKVEIK.

Preferably, the anti-CD3 antibody comprises a heavy chain amino acid sequence comprising:

(SEQ ID NO: 10)
QVQLVESGGGVVQPGRSLRLSCAASGFKFSGYGMHWVRQAPGKGLEWVAV
IWYDGSKKYYVDSVKGRFTISRDNSKNTLYLQMNSLRAEDTAVYYCARQM
GYWHFDLWGRGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPEAEGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK

and a light chain amino acid sequence comprising:

(SEQ ID NO: 11)
EIVLTQSPATLSLSPGERATLSCRASQSVSSYLAWYQQKPGQAPRLLIYD
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

This anti-CD3 antibody is referred to herein as NI-0401, Foralumab, or 28F11-AE. (See e.g., Dean Y, Dépis F, Kosco-Vilbois M. “Combination therapies in the context of anti-CD3 antibodies for the treatment of autoimmune diseases.” Swiss Med Wkly. (2012) (the contents of which are hereby incorporated by reference in its entirety).

In some embodiments, the anti-CD3 antibody is a fully human antibody or a humanized antibody. In some embodiments, the anti-CD3 antibody formulation includes a full length anti-CD3 antibody. In alternative embodiments, the anti-CD3 antibody formulation includes an antibody fragment that specifically binds CD3. In some embodiments, the anti-CD3 antibody formulation includes a combination of full-length anti-CD3 antibodies and antigen binding fragments that specifically bind CD3.

In some embodiments, the antibody or antigen-binding fragment thereof that binds CD3 is a monoclonal antibody, domain antibody, single chain, Fab fragment, a F(ab′)₂ fragment, a scFv, a scAb, a dAb, a single domain heavy chain antibody, or a single domain light chain antibody. In some embodiments, such an antibody or antigen-binding fragment thereof that binds CD3 is a mouse, other rodent, chimeric, humanized or fully human monoclonal antibody.

Optionally, the anti-CD3 antibody or antigen binding fragment thereof used in the formulations of the disclosure includes at least one an amino acid mutation. Typically, the mutation is in the constant region. The mutation results in an antibody that has an altered effector function. An effector function of an antibody is altered by altering, i.e., enhancing or reducing, the affinity of the antibody for an effector molecule such as an Fc receptor or a complement component. For example, the mutation results in an antibody that is capable of reducing cytokine release from a T-cell. For example, the mutation is in the heavy chain at amino acid residue 234, 235, 265, or 297 or combinations thereof. Preferably, the mutation results in an alanine residue at either position 234, 235, 265 or 297, or a glutamate residue at position 235, or a combination thereof.

Preferably, the anti-CD3 antibody provided herein contains one or more mutations that prevent heavy chain constant region-mediated release of one or more cytokine(s) in vivo.

In some embodiments, the anti-CD3 antibody or antigen binding fragment thereof used in the formulations of the disclosure is a fully human antibody. The fully human CD3 antibodies used herein include, for example, a L²³⁴L²³⁵→A²³⁴E²³⁵ mutation in the Fc region, such that cytokine release upon exposure to the anti-CD3 antibody is significantly reduced or eliminated. The L²³⁴L²³⁵→A²³⁴E²³⁵ mutation in the Fc region of the anti-CD3 antibodies provided herein reduces or eliminates cytokine release when the anti-CD3 antibodies are exposed to human leukocytes, whereas the mutations described below maintain significant cytokine release capacity. For example, a significant reduction in cytokine release is defined by comparing the release of cytokines upon exposure to the anti-CD3 antibody having a L²³⁴L²³⁵→A²³⁴E²³⁵ mutation in the Fc region to level of cytokine release upon exposure to another anti-CD3 antibody having one or more of the mutations described below. Other mutations in the Fc region include, for example, L²³⁴L²³⁵→A²³⁴A²³⁵, L²³⁵→E²³⁵, N²⁹⁷→A297, and D²⁶⁵→A²⁶⁵.

The term “cytokine” refers to all human cytokines known within the art that bind extracellular receptors expressed on the cell surface and thereby modulate cell function, including but not limited to IL-2, IFN-gamma, TNF-a, IL-4, IL-5, IL-6, IL-9, IL-10, and IL-13.

Pharmaceutical Compositions

The anti-CD3 antibodies described herein can be incorporated into a pharmaceutical composition suitable for oral or mucosal administration, e.g., by ingestion, inhalation, or absorption, e.g., via nasal, intranasal, pulmonary, buccal, sublingual, rectal, or vaginal administration. Such compositions can include an inert diluent or an edible carrier. For the purpose of oral therapeutic administration, the active compound (e.g., an anti-CD3 antibody) can be incorporated with excipients and used in solid or liquid (including gel) form. Oral anti-CD3 antibody compositions can also be prepared using an excipient. Pharmaceutically compatible binding agents, and/or adjuvant materials can be included as part of the composition. Oral dosage forms comprising anti-CD3 antibody are provided, wherein the dosage forms, upon oral administration, provide a therapeutically effective blood level of anti-CD3 antibody to a subject. Also provided are mucosal dosage forms comprising anti-CD3 antibody wherein the dosage forms, upon mucosal administration, provide a therapeutically effective blood level of anti-CD3 antibody to a subject. For the purpose of mucosal therapeutic administration, the active compound (e.g., an anti-CD3 antibody) can be incorporated with excipients or carriers suitable for administration by inhalation or absorption, e.g., via nasal sprays or drops, or rectal or vaginal suppositories.

Solid oral dosage forms include, but are not limited to, tablets (e.g., chewable tablets), capsules, caplets, powders, pellets, granules, powder in a sachet, enteric coated tablets, enteric coated beads, and enteric coated soft gel capsules. Also included are multi-layered tablets, wherein different layers can contain different drugs. Solid dosage forms also include powders, pellets and granules that are encapsulated. The powders, pellets, and granules can be coated, e.g., with a suitable polymer or a conventional coating material to achieve, for example, greater stability in the gastrointestinal tract, or to achieve a desired rate of release. In addition, a capsule comprising the powder, pellets or granules can be further coated. A tablet or caplet can be scored to facilitate division for ease in adjusting dosage as needed. The dosage forms of the present invention can be unit dosage forms wherein the dosage form is intended to deliver one therapeutic dose per administration, e.g., one tablet is equal to one dose. Such dosage forms can be prepared by methods of pharmacy well known to those skilled in the art (see Remington's Pharmaceutical Sciences, 18th ed., Mack Publishing, Easton Pa. (1990)).

Typical oral dosage forms can be prepared by combining the active ingredients in an intimate admixture with at least one excipient according to conventional pharmaceutical compounding techniques. Excipients can take a wide variety of forms depending on the form of preparation desired for administration. For example, excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents. Examples of excipients suitable for use in oral liquid dosage forms include, but are not limited to, water, glycols, oils, alcohols, flavoring agents, preservatives, and coloring agents.

Tablets and capsules represent convenient pharmaceutical compositions and oral dosage forms, in which case solid excipients are employed. If desired, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

As one example, a tablet can be prepared by compression or by molding. Compressed tablets can be prepared, e.g., by compressing, in a suitable machine, the active ingredients (e.g., an anti-CD3 antibody) in a free-flowing form such as powder or granules, optionally mixed with an excipient. Molded tablets can be made, e.g., by molding, in a suitable machine, a mixture of the powdered anti-CD3 antibody compound moistened, e.g., with an inert liquid diluent.

Excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gum tragacanth or gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidinones, methyl cellulose, pre-gelatinized starch, hydroxypropyl methyl cellulose, (e.g., Nos. 2208, 2906, 2910), microcrystalline cellulose, and mixtures thereof

Suitable forms of microcrystalline cellulose include, but are not limited to, the materials sold as AVICEL™ PH-101, AVICEL™ PH-103, AVICEL™. RC-581, AVICEL™PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL™ RC-581. Suitable anhydrous or low moisture excipients or additives include AVICEL™ PH-103 and Starch 1500™LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions and dosage forms of the invention is typically present in from about 50 to about 99 weight percent of the pharmaceutical composition or dosage form.

Disintegrants can be used in the pharmaceutical compositions and oral or mucosal dosage forms of the invention to provide tablets that disintegrate when exposed to an aqueous environment. Tablets containing too much disintegrant might disintegrate in storage, while those containing too little might not disintegrate at a desired rate or under desired conditions. Thus, a sufficient amount of disintegrant that is neither too much nor too little to detrimentally alter the release of the active ingredients should be used to form the pharmaceutical compositions and solid oral dosage forms described herein. The amount of disintegrant used varies based upon the type of formulation, and is readily discernible to those of ordinary skill in the art. Typically, pharmaceutical compositions and dosage forms comprise from about 0.5 to about 15 weight percent of disintegrant, preferably from about 1 to about 5 weight percent of disintegrant.

Disintegrants that can be used in pharmaceutical compositions and oral or mucosal dosage forms of the invention include, but are not limited to, agar-agar, alginic acid, calcium carbonate, Primogel, microcrystalline cellulose, croscarmellose sodium, crospovidone, polacrilin potassium, sodium starch glycolate, corn, potato or tapioca starch, other starches, pre-gelatinized starch, other starches, clays, other algins, other celluloses, gums, and mixtures thereof

Lubricants that can be used in pharmaceutical compositions and dosage forms of the invention include, but are not limited to, calcium stearate, magnesium stearate or Sterotes, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL™200, manufactured by W. R. Grace Co. of Baltimore, Md.), a coagulated aerosol of synthetic silica (marketed by Degussa Co. of Plano, Tex.), CAB-O-SIL™(a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated. A glidant such as colloidal silicon dioxide can also be used.

The pharmaceutical compositions and oral or mucosal dosage forms can further comprise one or more compounds that reduce the rate by which an active ingredient will decompose. Thus the oral dosage forms described herein can be processed into an immediate release or a sustained release dosage form. Immediate release dosage forms may release the anti-CD3 antibody in a fairly short time, for example, within a few minutes to within a few hours. Sustained release dosage forms may release the anti-CD3 antibody over a period of several hours, for example, up to 24 hours or longer, if desired. In either case, the delivery can be controlled to be substantially at a certain predetermined rate over the period of delivery. In some embodiments, the solid oral dosage forms can be coated with a polymeric or other known coating material(s) to achieve, for example, greater stability on the shelf or in the gastrointestinal tract, or to achieve control over drug release. Such coating techniques and materials used therein are known in the art. Such compounds, which are referred to herein as “stabilizers,” include, but are not limited to, antioxidants such as ascorbic acid and salt buffers. For example, cellulose acetate phthalate, polyvinyl acetate phthalate, hydroxypropylmethyl cellulose phthalate, methacrylic acid-methacrylic acid ester copolymers, cellulose acetate trimellitate, carboxymethylethyl cellulose, and hydroxypropylmethyl cellulose acetate succinate, among others, can be used to achieve enteric coating. Mixtures of waxes, shellac, zein, ethyl cellulose, acrylic resins, cellulose acetate, silicone elastomers can be used to achieve sustained release coating. See, for example, Remington, supra, Chapter 93, for other types of coatings, techniques and equipment.

Liquids for oral or mucosal administration represent another convenient dosage form, in which case a solvent can be employed. In some embodiments, the solvent is a buffered liquid such as phosphate buffered saline (PBS). Liquid oral dosage forms can be prepared by combining the active ingredient in a suitable solvent to form a solution, suspension, syrup, or elixir of the active ingredient in the liquid. The solutions, suspensions, syrups, and elixirs may optionally comprise other additives including, but not limited to, glycerin, sorbitol, propylene glycol, sugars or other sweeteners, flavoring agents, and stabilizers. Flavoring agents can include, but are not limited to peppermint, methyl salicylate, or orange flavoring. Sweeteners can include sugars, aspartame, saccharin, sodium cyclamate and xylitol.

In order to reduce the degree of inactivation of orally administered anti-CD3 antibody in the stomach of the treated subject, an antiacid can be administered simultaneously with the immunoglobulin, which neutralizes the otherwise acidic character of the gut. Thus in some embodiments, the anti-CD3 antibody is administered orally with an antacid, e.g., aluminum hydroxide or magnesium hydroxide such as MAALOX™antacid or MYLANTA™. antacid, or an H2 blocker, such as cimetidine or ranitidine. One of skill in the art will appreciate that the dose of antiacid administered in conjunction with an anti-CD3 antibody depends on the particular antacid used. When the antacid is MYLANTA™antacid in liquid form, between 15 ml and 30 ml can be administered, e.g., about 15 ml. When the cimetidine H2 blocker is used, between about 400 and 800 mg per day can be used.

The kits described herein can include an anti-CD3 antibody composition as an already prepared liquid oral or mucosal dosage form ready for administration or, alternatively, can include an anti-CD3 antibody composition as a solid pharmaceutical composition that can be reconstituted with a solvent to provide a liquid oral dosage form or mucosal dosage form. When the kit includes an anti-CD3 antibody composition as a solid pharmaceutical composition that can be reconstituted with a solvent to provide a liquid dosage form (e.g., for oral or nasal administration), the kit may optionally include a reconstituting solvent. In this case, the constituting or reconstituting solvent is combined with the active ingredient to provide a liquid oral dosage form of the active ingredient. Typically, the active ingredient is soluble in the solvent and forms a solution. The solvent can be, e.g., water, a non-aqueous liquid, or a combination of a non-aqueous component and an aqueous component. Suitable non-aqueous components include, but are not limited to oils; alcohols, such as ethanol; glycerin; and glycols, such as polyethylene glycol and propylene glycol. In some embodiments, the solvent is phosphate buffered saline (PBS).

For administration by inhalation, the mucosal anti-CD3 antibody compounds can be delivered in the form of an aerosol spray from pressured container or dispenser that contains a suitable propellant, e.g., a gas such as carbon dioxide, or a nebulizer. Such methods include those described in U.S. Pat. No. 6,468,798.

Systemic administration can also be by transmucosal means. For transmucosal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal drops or sprays, or rectal or vaginal suppositories.

The anti-CD3 antibody compounds can also be prepared in the form of suppositories (e.g., with conventional suppository bases such as cocoa butter and other glycerides) or retention enemas for rectal delivery.

In one embodiment, the oral or mucosal anti-CD3 antibody compositions are prepared with carriers that will protect the anti-CD3 antibody against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Dosage, toxicity and therapeutic efficacy of such anti-CD3 antibody compositions can be determined by standard pharmaceutical procedures in cell cultures (e.g., of cells taken from an animal after mucosal administration of an anti-CD3 antibody) or experimental animals, e.g., for determining the LD50 (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions which exhibit high therapeutic indices are preferred. While anti-CD3 antibody compositions that exhibit toxic side effects may be used, care should be taken to design a delivery system that targets such compounds to the site of affected tissue in order to minimize potential damage and, thereby, reduce side effects.

The data obtained from the cell cultures (e.g., of cells taken from an animal after mucosal administration of an anti-CD3 antibody) and animal studies can be used in formulating a range of dosage for use in humans. The dosage of anti-CD3 antibody compositions lies preferably within a range of circulating concentrations that include the ED5o with little or no toxicity. The dosage may vary within this range depending upon the dosage form employed and the route of administration utilized. For any oral or mucosal anti-CD3 antibody compositions used in the methods described herein, the therapeutically effective dose can be estimated initially from assays of cell cultures (e.g., of cells taken from an animal after mucosal administration of an anti-CD3 antibody). A dose may be formulated in animal models to achieve a desired circulating plasma concentration of IL-10 or TGFβ, or of regulatory cells, in the range that includes the IC₅₀ (i.e., the concentration of the test compound which achieves a half-maximal inhibition of symptoms) as determined in cell culture. Such information can be used to more accurately determine useful doses in humans. Levels of IL-10 or TGFβ. in plasma can be measured by methods known in the art, for example, by ELISA. Levels of regulatory cells can be measured by methods known in the art, for example, by flow cytometry-based methods.

As defined herein, a therapeutically effective amount of an anti-CD3 antibody (i.e., an effective dosage) depends on the antibody selected, the mode of delivery, and the condition to be treated. For instance, single dose amounts in the range of approximately 1:g/kg to 1000 g/kg may be administered; in some embodiments, about 5, 10, 50, 100, or 500:g/kg may be administered. In some embodiments, e.g., pediatric subjects, about 1 to 100:g/kg, e.g., about 25 or 50:g/kg, of anti-CD3 antibody can be administered. The anti-CD3 antibody compositions can be administered from one or more times per day to one or more times per week; including once every other day. The oral or mucosal anti-CD3 antibody compositions can be administered, e.g., for about 10 to 14 days or longer. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds can include a single treatment or, can include a series of treatments.

The oral or mucosal anti-CD3 antibody compositions can also include one or more therapeutic agents useful for treating an autoimmune disorder. Such therapeutic agents can include, e.g., NSAIDs (including COX-2 inhibitors); other antibodies, e.g., anti-cytokine antibodies, e.g., antibodies to IFN-.α-inverted., IFN γ and/or TNFαinverted.; gold-containing compounds; immunosuppressive drugs (such as corticosteroids, e.g., prednisolone and methyl prednisolone; cyclophosphamide; azathioprine; mycophenolate mofetil (MMF); cyclosporin and tacrolimus; methotrexate; or cotrimoxazole); heat shock proteins (e.g., as described in U.S. Pat. No. 6,007,821); and treatments for MS, e.g., .beta.-interferons (e.g., interferon β-1a, interferonβ1b), mitoxantrone, or glatiramer acetate.

The pharmaceutical compositions can be included in a container, pack, or dispenser together with instructions for administration.

Methods of Treatment and Prevention

The oral and mucosal anti-CD3 antibody compositions described herein can be administered to a subject to treat, prevent or alleviate a sign or symptom of disorders associated microglial activation.

Examples of disorders associated microglial activation include for example, a neurodegenerative disorder, an ischemic related disease or injury, traumatic brain injury or a lysosomal storage disease. Neurodegenerative disease include, but are not limited to, Alzheimer's Disease, Parkinson's Disease, Amyotrophic Lateral Sclerosis (ALS), and Huntington's Disease. Ischemic related disease but are not limited to, a ischemic-reperfusion injury, stroke, and myocardial infarction. The ischemic-reperfusion injury incudes injury to lung tissue, cardiac tissue or neuronal tissue. Traumatic brain injuries include, but are not limited to concussion such as is a repetitive concussive injury or whiplash. Lysosomal storage disease includes for example, Neimann-Pick disease.

A sign or symptom of a disease associated with microglial activation includes for example but not limited to amyloid plaque formation.

In some embodiments, a therapeutically effective amount of an oral or mucosal anti-CD3 antibody composition can be, e.g., the amount necessary to reduce microglial activation by about at least 20%. In some embodiments, microglial activation is reduced by at least about 30%, about 40%, about 50%, about 60%, about 70% about 80%, or about 90% from pre-treatment levels. In addition, concentrations of TGF-β1 can be measured. For example, TGF-β1 are measured in the peripheral blood, e.g., using an enzyme-linked immunosorbent assay (ELISA) or a cell-based assay such as FACS scanning, to monitor the induction of tolerance. In some embodiments, a therapeutically effective amount of an oral or mucosal anti-CD3 antibody composition is the amount necessary increase levels of cells secreting TGF-β1 by about 20% or more. In some embodiments, levels of cells secreting TGF-β1are increased by at least about 60%, 70%, 80%, 90%, or 100%, e.g., doubled.

In addition, cellular expression of CD74, H2-Ab and/ or CX3CR1 can be measured. In some embodiments, a therapeutically effective amount of an oral or mucosal anti-CD3 antibody composition is the amount necessary decrease the expression levels of CD74 and/or H2-Ab-1 by about 20% or more. In some embodiments, levels of expression of CD74 and/or H2-Ab-1 are decreased by at least about 60%, 70%, 80%, 90%, or 100%, e.g., half.

In some embodiments, a therapeutically effective amount of an oral or mucosal anti-CD3 antibody composition is the amount necessary increase the expression levels of CX3CR1 by about 20% or more. In some embodiments, levels of expression of CX3CR1 is increased by at least about 60%, 70%, 80%, 90%, or 100%, e.g., doubled

Furthermore, cellular expression of CX3CR1 and/or CCR2 on Ly6C^{high} splenocytes can be measured. In some embodiments, a therapeutically effective amount of an oral or mucosal anti-CD3 antibody composition is the amount necessary increases the expression levels of CX3CR1 and/or CCR2 on Ly6C^{high}splenocytes by about 20% or more. In some embodiments, levels of expression of CX3CR1 and/or CCR2on Ly6C^{high} splenocytes are increased by at least about 60%, 70%, 80%, 90%, or 100%, e.g., doubled.

Furthermore, expression of Hsp40 of Dusp1 byLy6C^{high} splenocytes can be measured. In some embodiments, a therapeutically effective amount of an oral or mucosal anti-CD3 antibody composition is the amount necessary increases the expression levels of Hsp40 of Duspl byLy6C^{high} splenocytes by about 20% or more. In some embodiments, levels of expression of Hsp40 of Dusp1 byLy6C^{high} splenocytes are increased by at least about 60%, 70%, 80%, 90%, or 100%, e.g., doubled.

The methods of treatment or prevention typically include administering to a subject an oral or mucosal anti-CD-3 antibody composition sufficient to stimulate the mucosal immune system. In some embodiments, the methods include administering an oral or mucosal anti-CD3 antibody composition sufficient to increase IL-10 and/or TGF-.beta. production by T cells in the peripheral blood, e.g., regulatory T cells, e.g., by about 100%, 200%, 300% or more. In some embodiments, the methods include administering an oral anti-CD3 antibody composition sufficient to decrease T cell proliferation in the peripheral blood, e.g., by about 20%; e.g., in some embodiments, by at least about 20%, 30%, 40%, 50%, 60%, 70%, 80%, 90%, or more.

Cytokine Release Syndrome (CRS) is not expected to be associated with orally or mucosal administered anti-CD3 antibodies, but the methods can include monitoring the subjects for signs and symptoms of Cytokine Release Syndrome, particularly after the first few doses but also after a treatment hiatus with resumption of therapy; such methods are particularly useful in determining the safety of oral or mucosal administration of the anti-CD3 antibodies. CRS is associated with arthralgias, myalgias, fevers, chills, hypoxia, nausea, and vomiting; severe cytokine release syndrome can cause pulmonary edema and suffocation. In some embodiments, the methods include lowering the subject's temperature to less than about 37.8° C. (100° F.) before the administration of any dose of the anti-CD3 antibody compositions. In some embodiments, the methods include screening the subject for clinical evidence of volume overload, uncontrolled hypertension, or uncompensated heart failure. In some embodiments, the methods include not administering the oral or mucosal anti-CD3 antibodies to subjects who have evidence of any of, volume overload, uncontrolled hypertension, or uncompensated heart failure. In some embodiments, the methods involve evaluating the subject's pulmonary function, and not administering the anti-CD3 antibodies to subjects who do not have a clear chest X-ray. In some embodiments, the methods include monitoring CD3+ T cell clearance and/or plasma levels of anti-CD3 antibody, and adjusting the dosage of the oral or mucosal anti-CD3 compositions accordingly.

In some embodiments, the methods include administering to the subject methylprednisolone sodium succinate 8.0 mg/kg, e.g., intravenously, e.g., 1 to 4 hours before administration of the oral or mucosal anti-CD3 antibody compositions. In some embodiments, the methods can include administering to the subject an anti-inflammatory agent, e.g., acetaminophen or antihistamine, before, concomitantly with, or after administration of the oral or mucosal anti-CD3 compositions.

In some embodiments, the methods include evaluating and/or monitoring a subject for anti-mouse antibodies, and discontinuing administration of the oral or mucosal anti-CD3 antibody compositions if the subject has anti-mouse antibody titers of greater than about 1:1000. The development of anti-mouse antibodies is not expected with orally or mucosally administered anti-CD3 antibodies.

In some embodiments, the oral or mucosal anti-CD3 antibody compositions are administered concurrently with one or more second therapeutic modalities, e.g., symptomatic treatment, high dose immunosuppressive therapy and/or autologous peripheral blood stem cell transplantation (HSCT). Such methods are known in the art and can include administration of agents useful for treating an autoimmune disorder, e.g., NSAIDs (including selective COX-2 inhibitors); other antibodies, e.g., anti-cytokine antibodies, e.g., antibodies to IFN-.α inverted., IFNγ, and/or TNFα-inverted.; gold-containing compounds; heat shock proteins (e.g., as described in U.S. Pat. No. 6,007,821); immunosuppressive drugs (such as corticosteroids, e.g., prednisolone and methyl prednisolone; cyclophosphamide; azathioprine; mycophenolate mofetil (MMF); cyclosporin and tacrolimus; methotrexate; or cotrimoxazole) and therapeutic cell preparations, e.g., subject-specific cell therapy, hematopoietic stem cell therapy. In some embodiments, the methods include administering one or more treatments for multiple sclerosis, e.g., .beta.-interferons (e.g., interferonβ1a, interferon β1b), mitoxantrone, or glatiramer acetate. In some embodiments, the methods include administering one or more non-anti-CD3 immunosuppressive drugs (such as corticosteroids, e.g., prednisolone and methyl prednisolone; cyclophosphamide; azathioprine; mycophenolate mofetil (MMF); cyclosporin and tacrolimus; methotrexate; or cotrimoxazole) to the subject, e.g., before, during, or after administration of the oral or mucosal anti-CD3 compositions.

### EXAMPLES

### Example 1

### Effect of Nasal ANTI-CD3 on Microglia in Young and Old Wild Type Mice

Young (2 months) and old (24 months) mice were treated with nasal anti-CD3 (aCD3) or isotype control (1 ug/5 ul) for 7 days daily. At day 8 mice were sacrificed and microglia were isolated. RNA was isolated and 50 ng were used for RNASeq. The transcripts per million (TPM) were used to generate heat maps of significantly changed genes using MultipleArrayViewer(MeV) software.

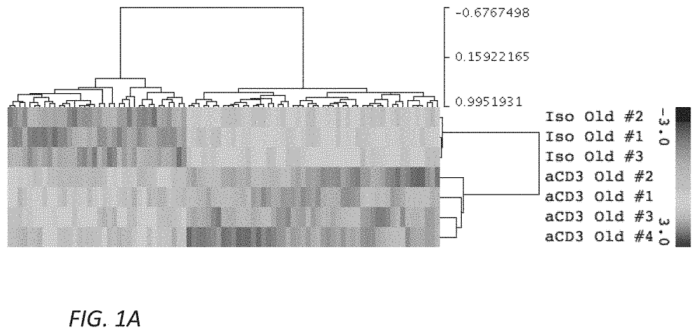

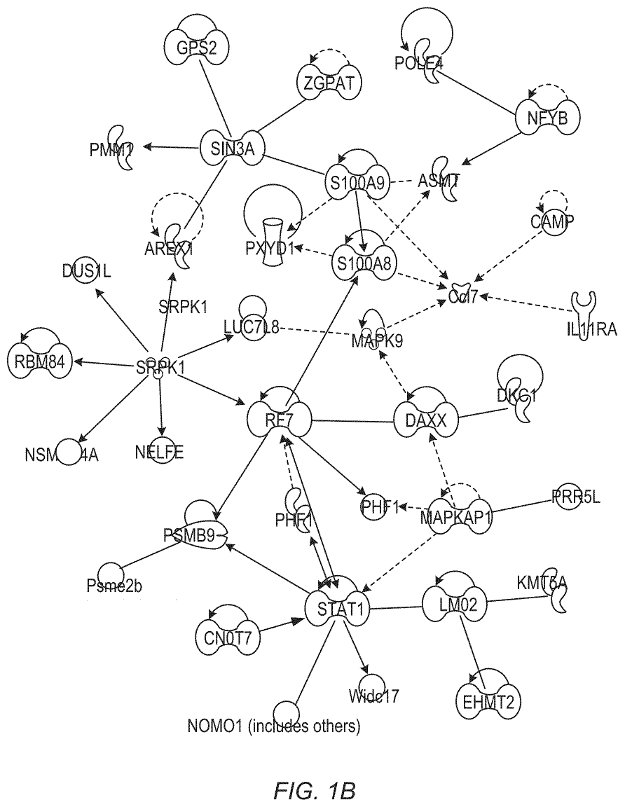

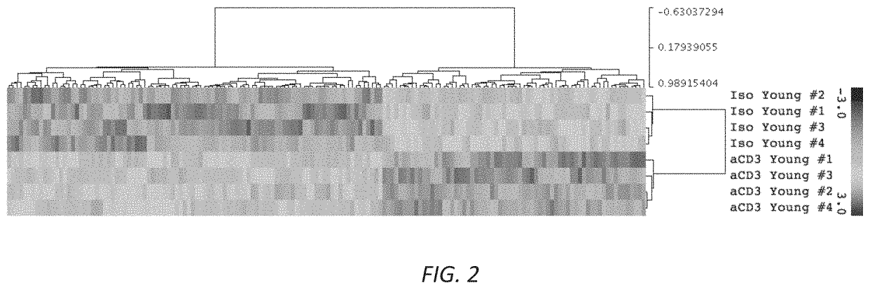

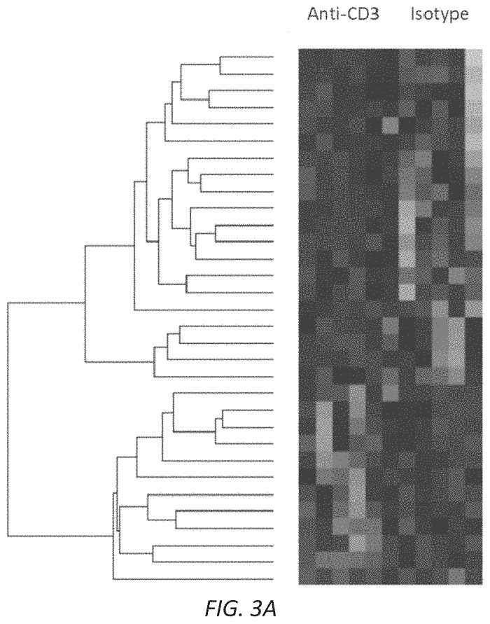

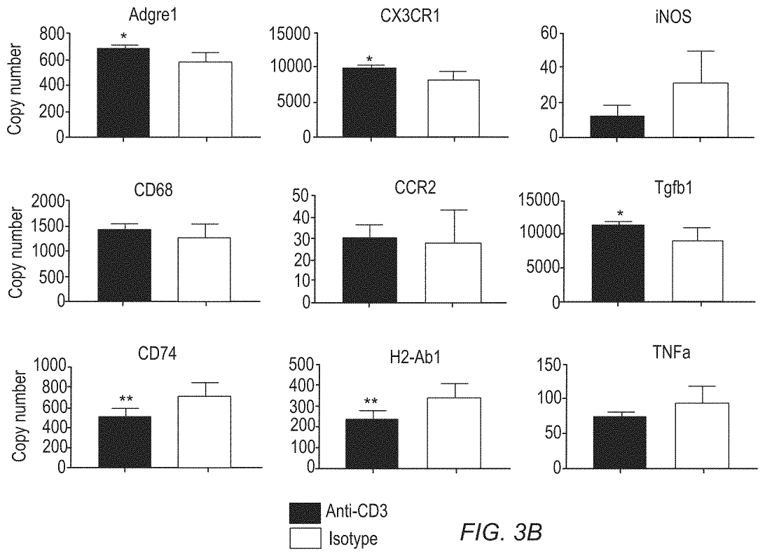

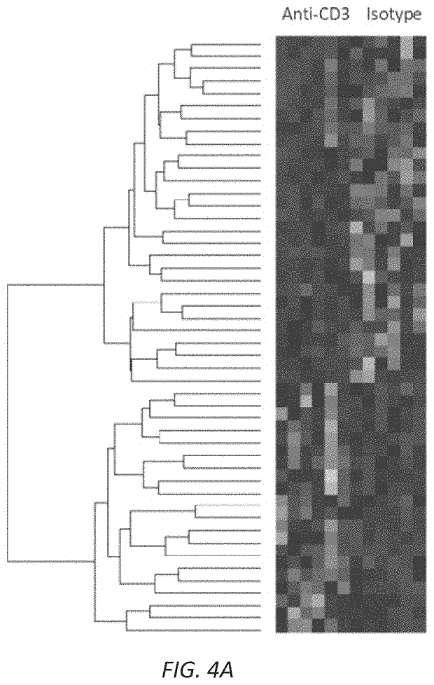

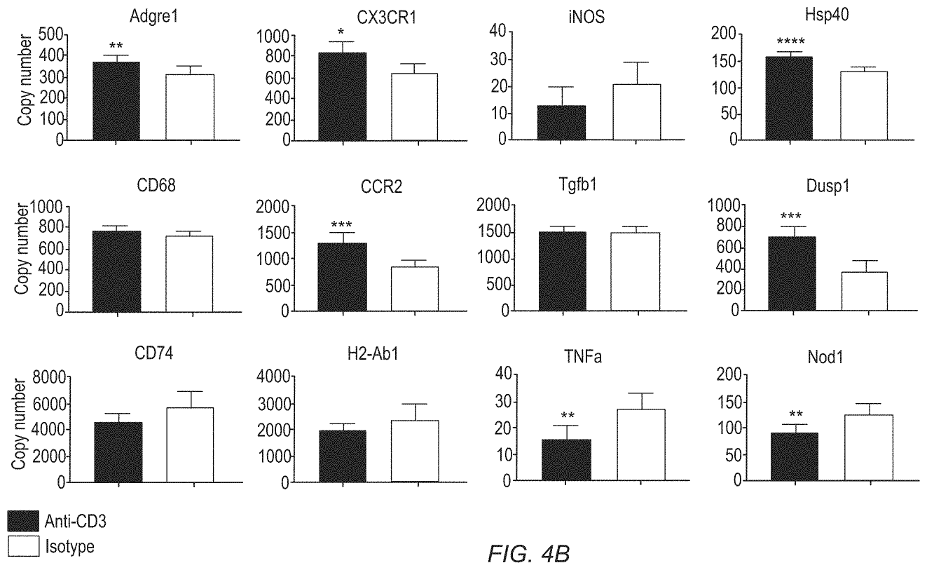

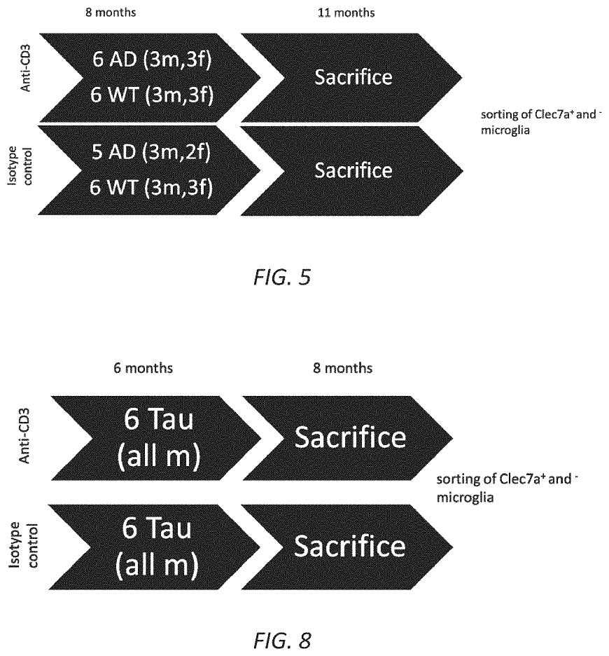

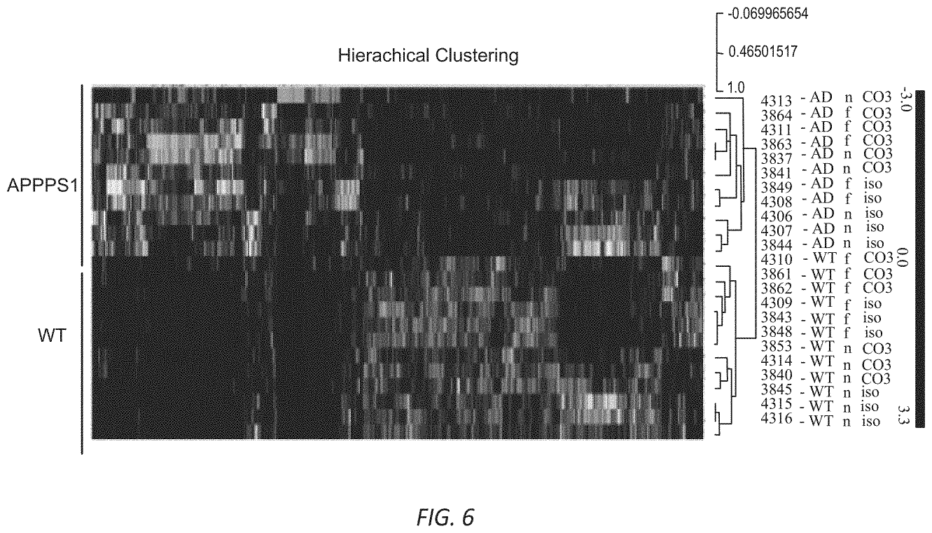

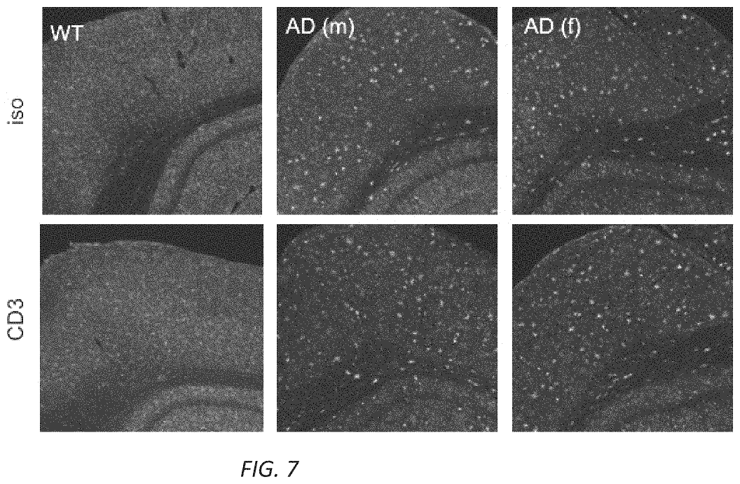

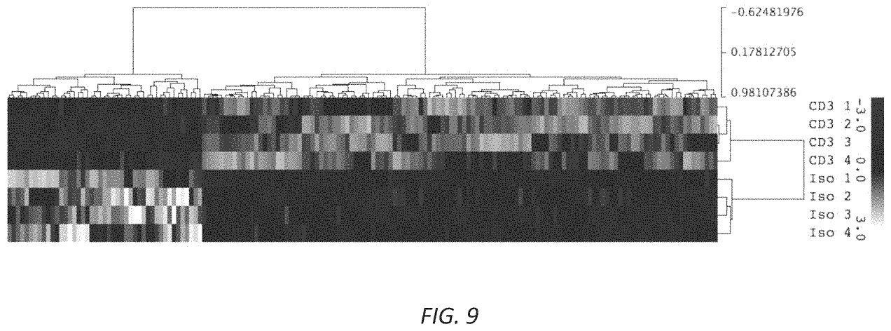

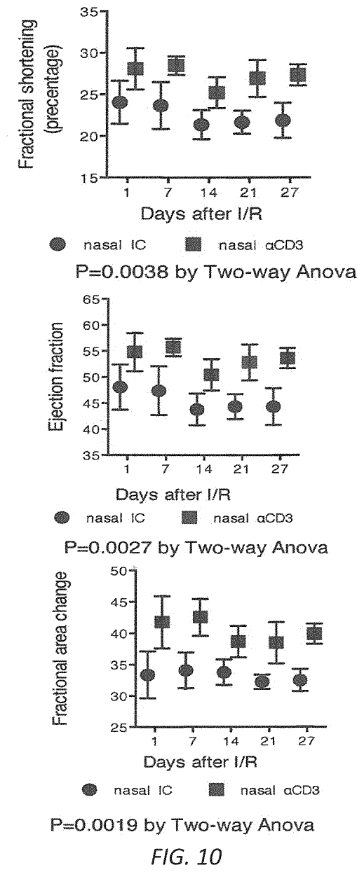

In young mice nasal CD3 significantly changes expression of 210 genes (FIG. 2). In old mice the same treatment induces expression changes in 116 genes (FIG. 1A). Thus, CD3 treatment affects microglial gene expression in young and old mice. However, the CD3 effect is different in young and old mice, with only 4 genes that are affected independent of the age of the mouse.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-21. (canceled)

22. A method of treating, preventing or alleviating a sign or symptom of traumatic brain injury or an ischemic related disease or injury, in a subject, the method comprising administering to the subject by intra-nasal administration a therapeutically effective amount of an anti-CD3 antibody, wherein the anti-CD3 antibody comprises a heavy chain complementarity determining region 1 (CDRH1) comprising the amino acid sequence GYGMH (SEQ ID NO: 1), a heavy chain complementarity determining region 2 (CDRH2) comprising the amino acid sequence VIWYDGSKKYYVDSVKG (SEQ ID NO: 3), a heavy chain complementarity determining region 3 (CDRH3) comprising the amino acid sequence QMGYWHFDL (SEQ ID NO: 4), a light chain complementarity determining region 1 (CDRL1) comprising the amino acid sequence RASQSVSSYLA (SEQ ID NO: 5), a light chain complementarity determining region 2 (CDRL2) comprising the amino acid sequence DASNRAT (SEQ ID NO: 6), and a light chain complementarity determining region 3 (CDRL3) comprising the amino acid sequence QQRSNWPPLT (SEQ ID NO: 7).

23. The method of claim 22, wherein the ischemic related disease or injury is ischemic reperfusion injury, stroke, global cerebral ischemia, or intracranial hemorrhage.

24. The method of claim 23, wherein the ischemic related disease or injury is intracranial hemorrhage

25. The method of claim 22, wherein the traumatic brain injury is a concussion, whiplash, or closed head injury.

26. The method of claim 25, where the concussion is a repetitive concussive injury.

27. The method of claim 22, wherein the anti-CD3 antibody comprises a variable heavy chain amino acid sequence comprising the amino acid sequence of SEQ ID NO: 8 and a variable light chain amino acid sequence comprising the amino acid sequence of SEQ ID NO: 9.

28. The method of claim 22, wherein the anti-CD3 antibody comprises a heavy chain amino acid sequence comprising the amino acid sequence of SEQ ID NO: 10 and a light chain amino acid sequence comprising the amino acid sequence of SEQ ID NO: 11.

29. The method of claim 22, wherein the anti-CD3 antibody is a monoclonal or polyclonal antibody.

30. The method of claim 22, wherein the anti-CD3 antibody is a fully human, humanized or chimeric.


SEQUENCE LISTING (11 sequences)

SEQ ID NO: 1 - protein, 5 aa, Artificial
  heavy chain complementarity determining region 1 (CDRH1)
GYGMH

SEQ ID NO: 2 - skipped

SEQ ID NO: 3 - protein, 17 aa, Artificial
  heavy chain complementarity determining region 2 (CDRH2)
VIWYDGSKKYYVDSVKG

SEQ ID NO: 4 - protein, 9 aa, Artificial
  heavy chain complementarity determining region 3 (CDRH3)
QMGYWHFDL

SEQ ID NO: 5 - protein, 11 aa, Artificial
  light chain complementarity determining region 1 (CDRL1)
RASQSVSSYLA

SEQ ID NO: 6 - protein, 7 aa, Artificial
  light chain complementarity determining region 2 (CDRL2)
DASNRAT

SEQ ID NO: 7 - protein, 10 aa, Artificial
  light chain complementarity determining region 3 (CDRL3)
QQRSNWPPLT

SEQ ID NO: 8 - protein, 118 aa, Artificial [sequence omitted]

SEQ ID NO: 9 - protein, 108 aa, Artificial [sequence omitted]

SEQ ID NO: 10 - protein, 448 aa, Artificial
  anti-CD3 antibody heavy chain
QVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 215 aa, Artificial
  anti-CD3 antibody light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC